SEQUENCE LISTING (22 sequences)

SEQ ID NO: 1 - DNA, 2653 bp, Artificial Sequence
  GCPII mutant (K699S)
ctcaaaaggggccggatttccttctcctggaggcagatgttgcctctctctctcgctcggattggttcagtgcactctagaaacactgctgtggtggagaaactggaccccaggtctggagcgaattccagcctgcagggctgataagcgaggcattagtgagattgagagagactttaccccgccgtggtggttggagggcgcgcagtagagcagcagcacaggcgcgggtcccgggaggccggctctgctcgcgccgagatgtggaatctccttcacgaaaccgactcggctgtggccaccgcgcgccgcccgcgctggctgtgcgctggggcgctggtgctggcgggtggcttctttctcctcggcttcctcttcgggtggtttataaaatcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaactcatatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagcctaagaggattctttagagaatccgtattgaatttgtgtggtatgtcactcagaaagaatcgtaatgggtatattgataaattttaaaattggtatatttgaaataaagttgaatattatatataaaaaaaaaaaaaaaaaaa

SEQ ID NO: 2 - protein, 750 aa, Artificial Sequence
  GCPII mutant (K699S) protein
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNSYAGESFPGIYDALFDIESKVDPSKAWGEVKRQIYVAAFTVQAAAETLSEVA

SEQ ID NO: 3 - DNA, 24 bp, Artificial Sequence
  primer F of hGCPII cDNA to amplify from whole cDNA via PCR
gatgtggaatctccttcacgaaac

SEQ ID NO: 4 - DNA, 26 bp, Artificial Sequence
  primer R of hGCPII cDNA to amplify from whole cDNA via PCR.
atcctcttaggctacttcactcaaag

SEQ ID NO: 5 - DNA, 30 bp, Artificial Sequence
  PrP-F
cctctttgtgactatgtggactgatgtcgg

SEQ ID NO: 6 - DNA, 28 bp, Artificial Sequence
  PrP-R
gtggataacccctcccccagcctagacc

SEQ ID NO: 7 - DNA, 24 bp, Artificial Sequence
  human APP-F
gactgaccactcgaccaggttctg

SEQ ID NO: 8 - DNA, 25 bp, Artificial Sequence
  human APP-R
cttgtaagttggattctcatatccg

SEQ ID NO: 9 - DNA, 33 bp, Artificial Sequence
  R210A-F
gggaagttttcgcgggaaataaggttaaaaatg

SEQ ID NO: 10 - DNA, 34 bp, Artificial Sequence
  R210A-R
catttttaaccttatttcccgcgaaaactttccc

SEQ ID NO: 11 - DNA, 34 bp, Artificial Sequence
  D387N-F
gtgtttggtggtattaatcctcagagtggagcag

SEQ ID NO: 12 - DNA, 34 bp, Artificial Sequence
  D387N-R
ctgctccactctgaggattaataccaccaaacac

SEQ ID NO: 13 - DNA, 34 bp, Artificial Sequence
  P388A-F
gtgtttggtggtattgacgctcagagtggagcag

SEQ ID NO: 14 - DNA, 34 bp, Artificial Sequence
  P388A-R
ctgctccactctgagcgtcaataccaccaaacac

SEQ ID NO: 15 - DNA, 33 bp, Artificial Sequence
  R536L-F
gcttcaggcagagctctgtatactaaaaattgg

SEQ ID NO: 16 - DNA, 33 bp, Artificial Sequence
  R536L-R
ccaatttttagtatacagagctctgcctgaagc

SEQ ID NO: 17 - DNA, 35 bp, Artificial Sequence
  G548P-F
gaaacaaacaattcagcccctatccactgtatcac

SEQ ID NO: 18 - DNA, 36 bp, Artificial Sequence
  G548P-R
gtgatacagtggataggggctgaatttgtttgtttc

SEQ ID NO: 19 - DNA, 36 bp, Artificial Sequence
  Y552I-F
cagcggctatccactgattcacagtgtctatgaaac

SEQ ID NO: 20 - DNA, 36 bp, Artificial Sequence
  Y552I-R
gtttcatagacactgtgaatcagtggatagccgctg

SEQ ID NO: 21 - DNA, 31 bp, Artificial Sequence
  K699S-F
caagcagccacaactcatatgcaggggagtc

SEQ ID NO: 22 - DNA, 31 bp, Artificial Sequence
  K699S-R
gactcccctgcatatgagttgtggctgcttg

(19) United States
(12) Patent Application Publication
(10) Pub. No.: US 2013/0089536 A1
(43) Pub. Date: Apr. 11, 2013

(54) PHARMACEUTICAL COMPOSITION TO PREVENT AND TREAT ALZHEIMER'S DISEASE COMPRISING GCP II MUTANT

(71) Applicant: Korea Center For Disease Control and Prevention, Chungcheongbuk-do (KR)

(72) Inventors: Hyunyoung KIM, Chungcheongbuk-do (KR); Suk Kyung LEE, Chungcheongbuk-do (KR); Sang Ick PARK, Chungcheongbuk-do (KR); Sangmee AHN, Chungcheongnam (KR)

(73) Assignee: KOREA CENTER FOR DISEASE CONTROL AND PREVENTION, Chungcheongbuk-do (KR)

(21) Appl. No.: 13/647,169

(22) Filed: Oct. 8, 2012

(30) Foreign Application Priority Data
Oct. 7, 2011 (KR) 1020110102610

(51) Int. Cl. A61K 38/48; A61P 25/28; A61P 25/00; C40B 30/04; C12Q 1/68; G01N 33/573; C12Q 1/37; G01N 27/62; A61K 31/711; A61P 7/02

(52) U.S. Cl. 424/94.63; 514/44 R; 435/6.13; 435/7.4; 435/6.12; 435/23; 506/9

(57) ABSTRACT

The present invention relates to a GCP II (glutamate carboxypeptidase II) mutant (K699S) having the activity of inhibiting glutamate production and the activity of cleavaging β-amyloid, and to a pharmaceutical composition for the prevention and treatment of a disease selected from the group consisting of amyloidosis, Alzheimer's disease, Down syndrome accompanying Alzheimer's disease, stroke, dementia, Huntington's disease, Pick's disease, and Creutzfeldt-Jakob disease comprising the GCP II mutant (K699S) as an active ingredient. The GCP II (glutamate carboxypeptidase II) mutant (K699S) demonstrates not only excellent Aβ cleavage activity compared with the wild type GCP II but also excellent activity of inhibiting glutamate production, unlike the wild type GCP II, so that the mutant has been confirmed to have higher effect and stability than the wild type, suggesting that the GCP II mutant can be effectively used for the prevention or treatment of neurodegenerative diseases.

### BACKGROUND OF THE INVENTION

1. Field of the Invention

The present invention relates to GCP II (glutamate carboxypeptidase II) mutant (K699S) having the activities of inhibiting glutamate production and cleavaging β-amyloid, and to a pharmaceutical composition for the prevention and treatment of disease selected from the group consisting of amyloidosis, Alzheimer's disease, Down syndrome accompanying Alzheimer's disease, stroke, dementia, Huntington's disease, Pick's disease, and Creutzfeid-Jacob disease comprising the GCP II mutant (K699S) as an active ingredient.

2. Description of the Related Art

GCP II is a type II integral membrane protein with an apparent molecular mass of 94-100 kDa, within the brain, resides primarily within glia, hydrolyzes extracellular NAAG (N-acetylaspartylglutamate) to NAA (N-acetylaspartate) and glutamate, and therefore impacts on glutamatergic transmission. Glutamate generated from NAAG hydrolysis is an essential factor in central nervous system acting as an excitatory neurotransmitter. However, once it is over-accumulated in the brain, it causes brain cell death and degeneration, resulting in degenerative brain disease such as Alzheimer's disease, stroke, dementia, Huntington's disease, Pick's disease, and Creutzfeid-Jakob disease (Choi, W. H., Oh, Y. S., Ahn, J. Y., Kim, S. R. and Ha, T. Y. (2005) Antioxidative and protective effects of Ulmus davidiana var. japonica extracts on glutamate-induced cytotoxicity in PC12 cells. Korean J. Food Sci. Technol. 37, 479-483; Jeon, H. J., Park, S. W. and Mun, B. S. (2004) Effects of Gwibitang on glutamate-induced death in rat neonatal astrocytes. J Korean Oriental Med. 25, 184-193; Lee, J., Kim, M. S., Lee, C, Kim, H. Y., Choi, D. H., Kim, T. Y., Son, Y. and Park, R. (2003) Role of morphine in the glutamate-induced oxidative damage of C6 glial cells. Korean J Anesthesiol. 45, 271-277).

GCP II has several names for its enzymatic action and expression area. Because of its strong expression in the prostate (where its function is unknown), it is named as PSMA; in central nervous system, where it metabolizes the brain neurotransmitter, N-acetylaspartyl-glutamate, it is named NAALADase; in the proximal small intestine, where its role is removing gamma-linked glutamates from poly-g-glutamated folate, folate hydrolase FOLH1, and as a carboxypeptidase, it is called glutamate carboxypeptidase II, GCP II.

The structure of GCP II also provides insight into its catalytic mechanism and substrate specificity. GCP II has a binuclear Zn²⁺ centre at the active site in which two zinc atoms share a bridging carboxylate ligand. The α-NAAG binds to the glutamate side chain to form electrostatic interactions with the arginine patch of GCP II, and the oxygen from the carbonyl to be attacked and the C-terminus binding to the zinc ions. The remainder of the substrate (e.g., NAA in α-NAAG) is accommodated by the substrate-binding cavity with specific interactions with Arg-210. Larger substrates ((e.g., poly-γ-glutamated folates with up to four γ-linked glutamates) (9)) require some structural rearrangements to avoid steric clashes with the pocket and could have an alternate mode of binding. Also GCP II has two active sites, S1′ pocket and S1 pocket (Mlcochova P., Plechanovova A., Barinka C., Mahadevan D., Saldanha J. W., Rjlisek L., Konvalinka J. (2007) Mapping of the active site of glutamate carboxypeptidase II by site-directed mutagenesis. FEBS J. 274 (18): 4731-41). The S1′ pocket contributes the high affinity binding of NAAG and GCP II inhibitors, whereas the S1 pocket acts as a ‘fine-tuning’ of GCP II substrate specificity.

Until now, the main function of GCP II is known for NAAG hydrolysis action. Therefore, the potent and selective GCP II inhibitor, 2-PMPA (2-(Phosphonomethyl) pentanedioic acid, has been proved to decrease brain glutamate and provide neuroprotection in preclinical models of stroke, amyotrophic lateral sclerosis, and neuropathic pain.

In previous studies, it has been reported that GCP II could be used for the treatment of Alzheimer's disease and Down syndrome by eliminating or preventing the accumulation of Aβ (amyloid β) in the brain through Aβ hydrolysis action (Korean Patent Publication No: 10-2007-0023832). However, according to the previous studies, glutamate produced from NAAG hydrolysis mediated by GCP II is known to cause not only neurotoxicity but also brain cell death and degeneration, once it is over-accumulated in the brain which can be a reason for degenerative brain disease such as Alzheimer's disease, stroke, dementia, Huntington's disease, Pick's disease, and Creutzfeid-Jakob disease. Therefore, it was very difficult to guarantee the stability of GCP II in use. So, a novel method to overcome the said problem has been requested.

The inventors of the present invention have studied to develop a novel method to overcome the above-mentioned problem. As a result, the inventors produced various GCP II mutants and analyzed the activity of each mutant. The inventors of the present invention finally completed this invention by confirming that the GCP II mutant, K699S, has better Aβ cleavage activity than wild type GCP II and also has the inhibitory activity of glutamate generation which is the activity that wild type GCP II does not have, suggesting that the mutant can be effectively used for the prevention or treatment of disease selected from the group consisting of amyloidosis, Alzheimer's disease, Down syndrome accompanying Alzheimer's disease, stroke, dementia, Huntington's disease, Pick's disease, and Creutzfeid-Jakob disease, since it has higher effect and stability than wild type GCP II has.

### SUMMARY OF THE INVENTION

An object of the present invention is to provide GCP II (glutamate carboxypeptidase II) mutant (K699S) having the activities of inhibiting glutamate production and degrading β-amyloid.

Another object of the present invention is to provide a preventive or therapeutic agent for the disease selected from the group consisting of amyloidosis, Alzheimer's disease, Down syndrome accompanying Alzheimer's disease, stroke, dementia, Huntington's disease, Pick's disease, and Creutzfeid-Jakob disease by using the said GCP II mutant (K699S).

Further object of the present invention is to provide a screening method for a preventive or therapeutic agent for the disease selected from the group consisting of amyloidosis, Alzheimer's disease, Down syndrome accompanying Alzheimer's disease, stroke, dementia, Huntington's disease, Pick's disease, and Creutzfeid-Jakob disease by using the said GCP II mutant (K699S).

To achieve the above objects, the present invention provides a pharmaceutical composition for the prevention and treatment of disease selected from the group consisting of amyloidosis, Alzheimer's disease, Down syndrome accompanying Alzheimer's disease, stroke, dementia, Huntington's disease, Pick's disease, and Creutzfeid-Jakob disease that comprises GCP II mutant prepared by replacing the 699^{th} amino acid from N-terminal of the total amino acid sequence of GCP II (glutamate carboxypeptidase II) (Lysine, K) with Serine (S) as an active ingredient.

The present invention also provides a preventive and therapeutic agent for the disease selected from the group consisting of amyloidosis, Alzheimer's disease, Down syndrome accompanying Alzheimer's disease, stroke, dementia, Huntington's disease, Pick's disease, and Creutzfeid-Jakob disease that comprises an expression vector expressing the GCP II mutant prepared by replacing the 699^{th} amino acid from N-terminal of the total amino acid sequence of GCP II (Lysine, K) with Serine (S) as an active ingredient.

The present invention further provides a pharmaceutical composition for the screening of a preventive and therapeutic agent for the disease selected from the group consisting of amyloidosis, Alzheimer's disease, Down syndrome accompanying Alzheimer's disease, stroke, dementia, Huntington's disease, Pick's disease, and Creutzfeid-Jakob disease that comprises GCP II mutant gene prepared by replacing the 699^{th} amino acid from N-terminal of the total amino acid sequence of GCP II (Lysine, K) with Serine (S) as an active ingredient.

The present invention further provides a pharmaceutical composition for the screening of a preventive and therapeutic agent for the disease selected from the group consisting of amyloidosis, Alzheimer's disease, Down syndrome accompanying Alzheimer's disease, stroke, dementia, Huntington's disease, Pick's disease, and Creutzfeid-Jakob disease that comprises GCP II mutant protein prepared by replacing the 699^{th} amino acid from N-terminal of the total amino acid sequence of GCP II (Lysine, K) with Serine (S) as an active ingredient.

The present invention also provides a screening method of a candidate material for the prevention and treatment of disease selected from the group consisting of amyloidosis, Alzheimer's disease, Down syndrome accompanying Alzheimer's disease, stroke, dementia, Huntington's disease, Pick's disease, and Creutzfeid-Jakob disease comprising steps of:

1) treating test samples to the cells expressing GCP II mutant prepared by replacing the 699^{th} amino acid from N-terminal of the total amino acid sequence of GCP II (Lysine, K) with Serine (S);

2) measuring the expression level of GCP II mutant protein in the cells of step 1); and

3) selecting a test sample that reduces the expression level of GCP II mutant protein of step 2), compared with the control group not treated with any test sample.

In addition, the present invention provides a screening method of a candidate material for the prevention and treatment of disease selected from the group consisting of amyloidosis, Alzheimer's disease, Down syndrome accompanying Alzheimer's disease, stroke, dementia, Huntington's disease, Pick's disease, and Creutzfeid-Jakob disease comprising steps of:

1) treating test samples to GCP II mutant protein prepared by replacing the 699^{th} amino acid from N-terminal of the total amino acid sequence of GCP II (Lysine, K) with Serine (S)

2) measuring the activity of GCP II mutant protein of step 1); and

3) selecting a test sample that reduces the activity of GCP II mutant protein of step 2), compared with the control group not treated with any test sample.

### ADVANTAGEOUS EFFECT

As explained hereinbefore, the GCP II (glutamate carboxypeptidase II) mutant (K699S) of the present invention not only demonstrated excellent Aβ cleavage activity, compared with the wild type GCP II, but also showed inhibitory activity of glutamate production which is something that the wild type GCP II did not have. Therefore, the GCP II mutant of the present invention can be effectively used for the development of a preventive or therapeutic agent for the disease selected from the group consisting of amyloidosis, Alzheimer's disease, Down syndrome accompanying Alzheimer's disease, stroke, dementia, Huntington's disease, Pick's disease, and Creutzfeid-Jakob disease since it has much better effect and has excellent stability, compared with the wild type GCP II.

### BRIEF DESCRIPTION OF THE DRAWINGS

The application of the preferred embodiments of the present invention is best understood with reference to the accompanying drawings, wherein:

FIG. 1 is a set of graphs illustrating the effect of 2-PMPA on the degradation of Aβ and NAAG.

(a, b) rhGCP II was incubated with Aβ₄₀ or NAAG with or without 2-PMPA or EDTA. A(340 or NAAG level was detected by ELISA or NAAG cleavage assay. (c) After lentiviral GCP II was infected into rat primary astrocytes, monomeric Aβ₄₀ was treated to cells. And 2-PMPA was treated thereto at various doses. The Aβ₄₀ concentration in cell growth medium was measured by ELISA assay. (d, e) Eight-month old APP Swedish/PS1E9 transgenic mice were injected intraperitoneally with 10 mg/kg of 2-PMPA or PBS 2/wk for 1 month (9 times). Membrane fractions were analyzed by NAAG and ELISA assay.

FIG. 2 is a set of diagrams illustrating the site-directed mutagenesis of GCP II.

(a) Sequence of primers were used for site-directed mutagenesis. (b) Expressions of various mutant proteins in transfected HEK 293 cells.

FIG. 3 is a set of graphs and photographs illustrating the NAAG cleavage activity in S1′ pocket mutants of GCP II.

(a) Mutant proteins with a substitution in the S1′ pocket (R210A, K699S) did not cleave N-acetyl-aspartyl-glutamate (NAAG). But, other proteins with a mutation in the SI pocket (R536L, R548P) maintained the NAAG cleavage activity about 40%. (b) NAAG cleavage activity of GCP II was not affected by Aβ peptide.

FIG. 4 is a set of graphs and photographs illustrating the effect of GCP II mutant on Aβ cleavage.

(a, b) In H4 cells, over-expressed S1′ mutant (K699S) cleaved amyloid-β40, but S1 mutant (G548P) did not. (c) The remaining A(3 was reduced not by G548P but by K699S.

### DESCRIPTION OF THE PREFERRED EMBODIMENTS

Hereinafter, the present invention is described in detail.

The present invention provides GCP II (glutamate carboxypeptidase II) mutant (K699S).

The said GCP II mutant is characterized by the replacement of the 699^{th} amino acid (Lysine, K) from N-terminal of the total amino acid sequence of GCP II with Serine (S), more precisely, by having the amino acid sequence represented by SEQ ID. NO: 2, but not always limited thereto.

The said GCP II mutant is characterized by having the activities of inhibiting glutamate production and degrading β-amyloid simultaneously, but not always limited thereto.

The present invention also provides a pharmaceutical composition for the prevention and treatment of disease selected from the group consisting of amyloidosis, Alzheimer's disease, Down syndrome accompanying Alzheimer's disease, stroke, dementia, Huntington's disease, Pick's disease, and Creutzfeid-Jakob disease that comprises GCP II mutant prepared by replacing the 699^{th} amino acid from N-terminal of the total amino acid sequence of GCP II (glutamate carboxypeptidase II) (Lysine, K) with Serine (S) as an active ingredient.

The said GCP II mutant preferably has the amino acid sequence represented by SEQ ID. NO: 2, but not always limited thereto.

The said GCP II mutant preferably has the activities of inhibiting glutamate production and degrading β-amyloid simultaneously, but not always limited thereto.

The β-amyloid is preferably soluble or insoluble β-amyloid, but not always limited thereto.

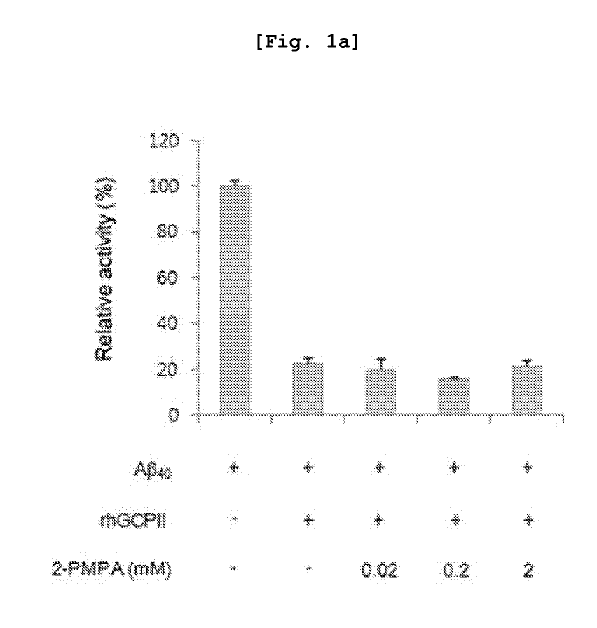

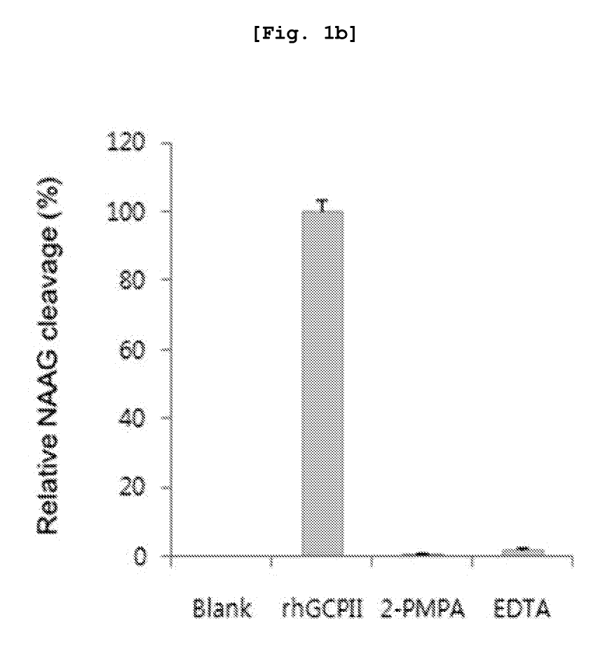

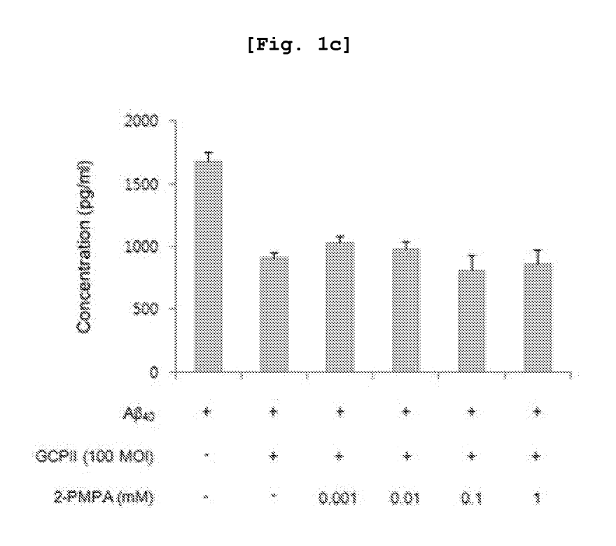

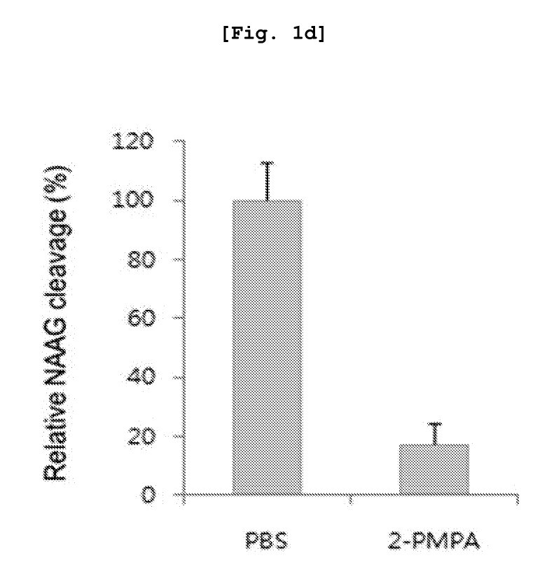

To confirm the effect of 2-PMPA, a specific inhibitor of GCP II, on Aβ peptides, recombinant human GCPII (rhGCP II) was incubated with Aβ₁₋₄₀, to which 2-PMPA was added at the different concentrations and analyzed by ELISA. As a result, rhGCP II cleaved Aβ₁₋₄₀ peptides about 80 percent compared with the control, but 2-PMPA had no effect on Aβ₁₋₄₀ cleavage of GCP II (see FIG. 1a). It was also confirmed that 2-PMPA did not block Aβ₁₋₄₀ cleavage of lentiviral GCP II in primary rat astrocytes (see FIG. 1c). Hydrolysis of NAAG by GCP II was completely blocked by 2-PMPA (see FIG. 1b). To investigate the effect of 2-PMPA in the mouse brain, 2-PMPA was treated into transgenic AD model mice. The inventors of the present invention used 2-PMPA (10 mg/kg, intraperitoneally, 2×/wk for 1 month) to treat 8-month old APP Swedish/PS1Δ9 transgenic mice. Aβ₁₋₄₀ and Aβ₁₋₄₂ levels in the cortical membrane fractions were analyzed by ELISA and NAAG cleavage activity of GCP II was measured by NAAG assay. As a result, cleavage of NAAG was reduced about 90 percent in 2-PMPA treated mouse compared with PBS control (see FIG. 1d). However, total levels of Aβ₁₋₄₀ and Aβ₁₋₄₂ were not changed by the treatment of 2-PMPA (see FIG. 1e). Therefore, it was suggested that NAAG or Aβ cleavage site in GCP II was different.

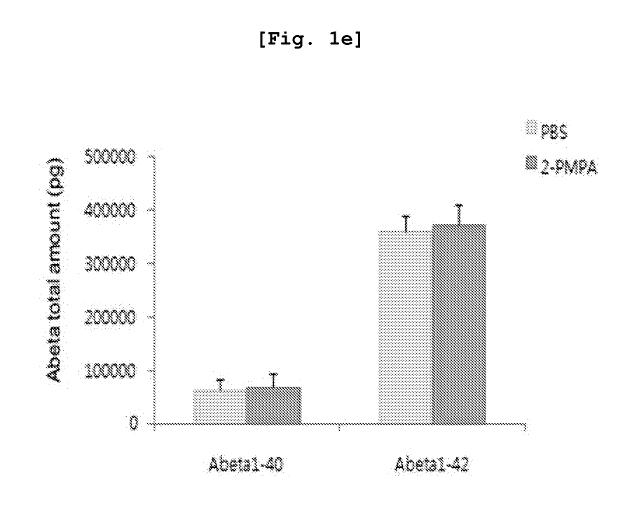

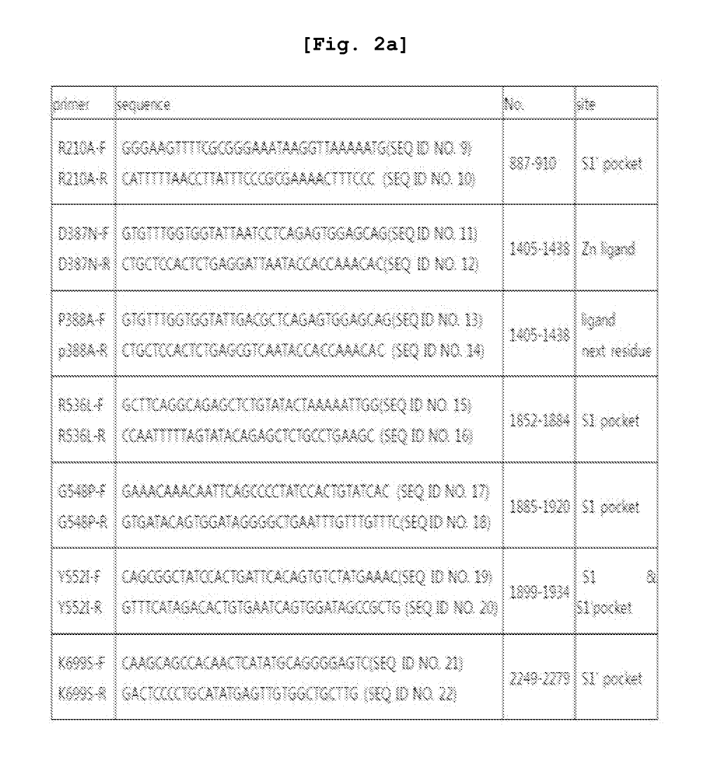

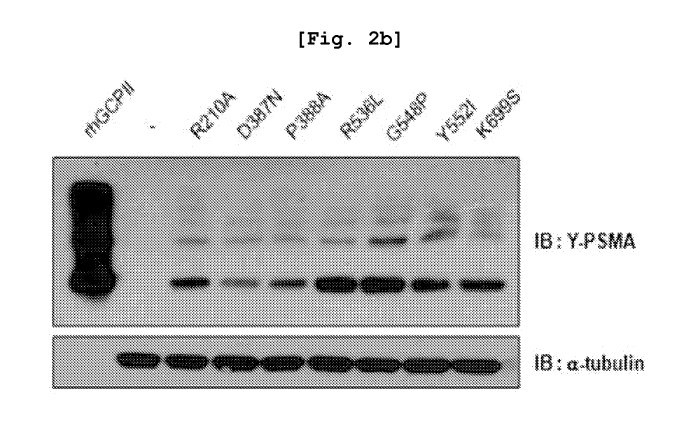

Based on the fact that GCP II is composed of two active sites which are called S1 and S1′pocket, a variety of GCP II [ref] mutations, precisely 7 GCP II mutations (R210A, D387N, P388A, R536L, G548P, Y552I, K699S) were constructed (see FIG. 2a). Using two complementary oligonucleotide primers having each mutation, amino acids were changed by site-directed mutagenesis. To determine the expression of various GCP II having mutation, mutant plasmids were transfected into HEK293T cells. Then, expression pattern was confirmed by western blotting. As a result, the pattern of bands was similar to that of rhGCP II and one of the bands was predominately seen at 98 kDa which is the predicted size of GCP II (see FIG. 2b).

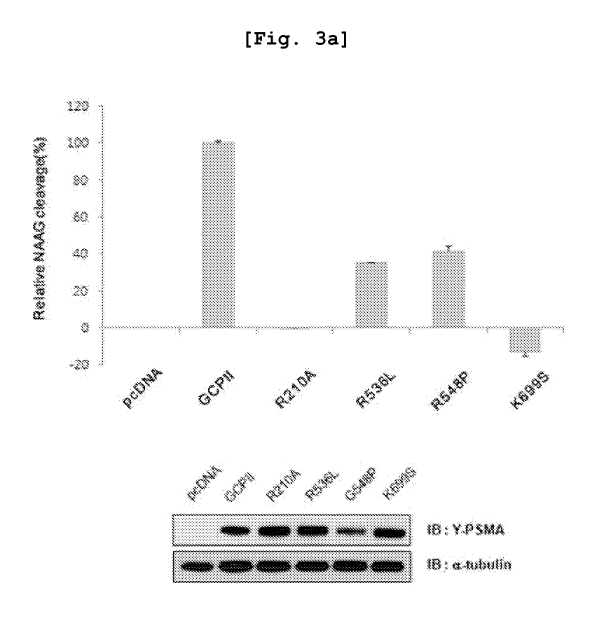

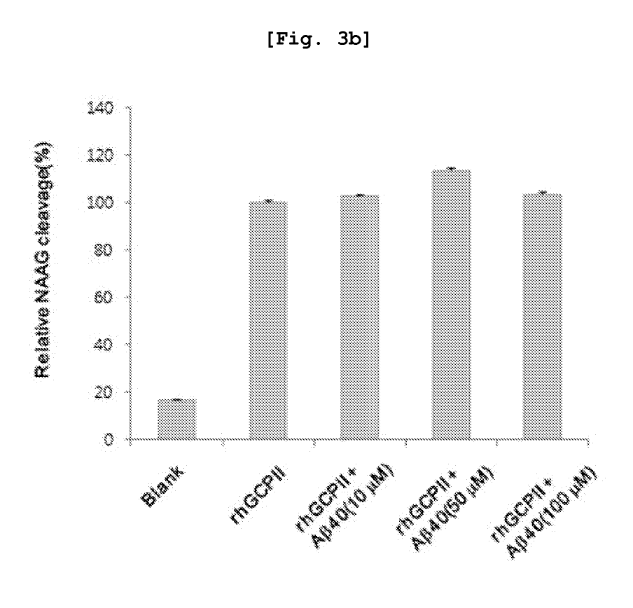

The inventors of the present invention also investigated the changes of enzyme activity of GCP II mutant. To analyze enzyme activity of various mutants, mutant proteins with a substitution in the S1′ pocket (R210A, K699S) or S1 pocket (R536L, G548P) were transfected in H4 cells. To measure NAAG hydrolysis, cell lysate was incubated with [H]³-labeled NAAG (20 μM) and GCP II specific substrate. As a result, it was confirmed that the S1′ pocket mutants (R210A, K699S) completely lost their activity which is hydrolysis of NAAG, while the SI pocket mutants (R536L, G548P) maintained their enzyme activity about 40% compared with wild type GCP II (see FIG. 3a). Additionally, to examine the effect of Aβ on NAAG cleavage of GCP II, rhGCP II was incubated with [H]³-labeled NAAG (20 μM) and Aβ peptides at the same time. As a result, NAAG cleavage activity of GCP II was not affected by Aβ peptide (see FIG. 3b). Therefore, it was confirmed that hydrolysis of NAAG in S1′ pocket was not affected by Aβ, indicating that the mechanism of NAAG cleavage in GCP II was different from that of Aβ cleavage therein.

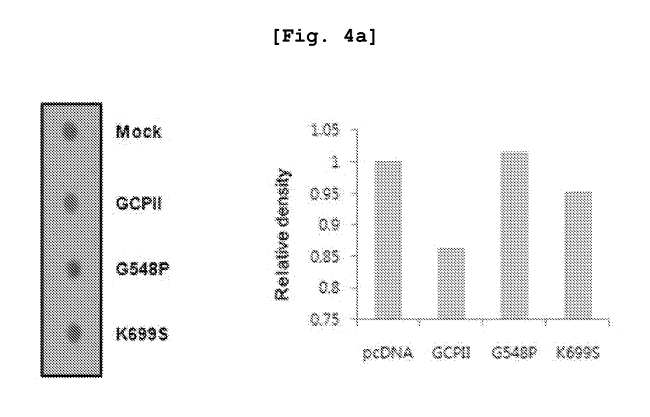

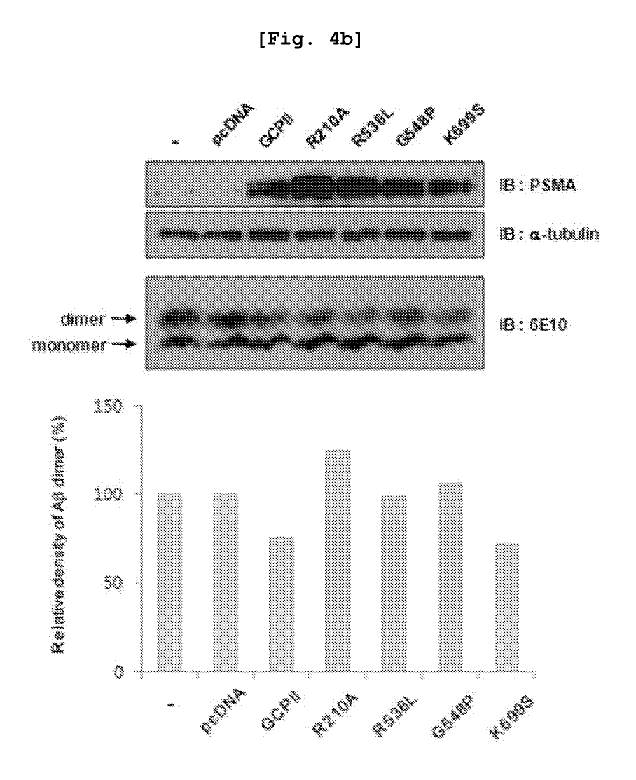

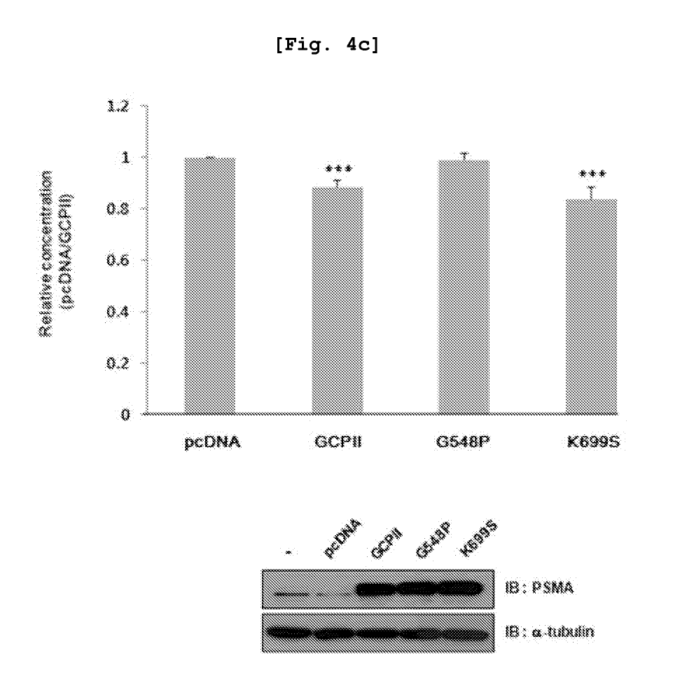

To identify Aβ clearance activity of GCP II mutants, various mutant plasmids were transfected in PC3 cells known not to express endogenous GCP II. After 48 hr, the cell lysates were mixed with Aβ (2 μM) and incubated for overnight. The remaining Aβ peptides were detected by dot blotting and western blotting using 6E10 antibody. As a result, the K699S mutant cleaved Aβ more than wild type but the G548P mutant lost its Aβ clearance activity (see FIGS. 4a and 4b). The inventors of the present invention also measured cleavage activity of GCP II on exogenously treated Aβ peptides in culture medium by ELISA. One day after transfection, 1 ng/ml Aβ was treated into the medium, followed by incubation for 8 hr at 37° C. Similarly with western blotting, residual Aβ was reduced by K699S but not by G548P (see FIG. 4c). Therefore, it was confirmed that SI pocket plays a more important role in Aβ cleavage than S1′ pocket, suggesting that NAAG hydrolysis and Aβ cleavage activities are varied from the type of GCP II enzyme.

The GCP II (glutamate carboxypeptidase II) mutant K699S of the present invention not only has excellent Aβ cleavage activity but also overcomes the stability problem that the wild type GCP II has, so that it can be effectively used for the treatment of disease selected from the group consisting of amyloidosis, Alzheimer's disease, Down syndrome accompanying Alzheimer's disease, stroke, dementia, Huntington's disease, Pick's disease, and Creutzfeid-Jakob disease since it can control simultaneously the production and inhibition of glutamate generated from NAAG hydrolysis and causing neurotoxicity.

The composition of the present invention comprising the GCP II mutant as an active ingredient can additionally include pharmaceutically and physiologically acceptable adjuvants, in addition to the said active ingredient, which are exemplified by solubilizing agents such as excipients, disintegrating agents, sweetening agents, binding agents, coating agents, inflating agents, lubricants, slip modifiers, or flavoring agents.

The composition of the present invention comprising the GCP II mutant protein as an active ingredient can additionally include pharmaceutically acceptable carriers, in addition to the said active ingredient, for the preparation of a pharmaceutical composition.

The pharmaceutically acceptable carrier can be selected or be prepared by mixing more than one ingredients selected from the group consisting of saline, sterilized water, Ringer's solution, buffered saline, dextrose solution, maltodextrose solution, glycerol and ethanol. Other general additives such as anti-oxidative agent, buffer solution, bacteriostatic agent, etc., can be added. In order to prepare injectable solutions such as aqueous solution, suspension and emulsion, diluents, dispersing agents, surfactants, binders and lubricants can be additionally added. The composition of the present invention can further be prepared in suitable forms for each disease or according to ingredients by following a method represented in Remington's Pharmaceutical Science (Mack Publishing Company, Easton Pa.).

The composition of the present invention comprising the GCP II mutant protein as an active ingredient can be prepared as granules, powders, coated tablets, capsules, suppositories, syrups, solutions, suspensions, emulsions, drops, or injectable solutions and sustained-release preparations.

The composition of the present invention comprising the GCP II mutant protein as an active ingredient can be administered by any conventional pathway that can deliver the drug into a target area, particularly by local, oral, parenteral, intranasal, intravenous, intramuscular, hypodermic, ophthalmic or transdermal administration.

The effective dosage of the composition of the present invention comprising the GCP II mutant protein as an active ingredient can be determined according to various factors such as a kind of disease, severity of disease, active ingredients and other constituents of a composition, a form of preparation, weight, age, gender, health condition, diet, administration frequency, administration method, excretion, and treatment time, etc. The preferable dosage of the GCP II mutant protein is 0.1 μg/kg˜0.1 g/kg per day, and administration frequency is once a day or preferably a few times a day.

The composition of the present invention can be administered alone or treated together with hormone therapy, drug treatment and biological regulators for the treatment of disease selected from the group consisting of amyloidosis, Alzheimer's disease, Down syndrome accompanying Alzheimer's disease, stroke, dementia, Huntington's disease, Pick's disease, and Creutzfeid-Jakob disease.

The present invention also provides a preventive and therapeutic agent for the disease selected from the group consisting of amyloidosis, Alzheimer's disease, Down syndrome accompanying Alzheimer's disease, stroke, dementia, Huntington's disease, Pick's disease, and Creutzfeid-Jakob disease that comprises expression vector expressing the GCP II mutant prepared by replacing the 699th amino acid from N-terminal of the total amino acid sequence of GCP II (Lysine, K) with Serine (S) as an active ingredient.

The expression vector herein is a viral expression vector selected from the group consisting of adenovirus, adeno-associated virus, retrovirus, and vaccinia virus, but not always limited thereto.

The GCP II (glutamate carboxypeptidase II) mutant K699S of the present invention not only has excellent Aβ cleavage activity but also overcomes the stability problem that the wild type GCP II has, so that the composition comprising the expression vector expressing the GCP II mutant as an active ingredient can be effectively used for the prevention and treatment of disease selected from the group consisting of amyloidosis, Alzheimer's disease, Down syndrome accompanying Alzheimer's disease, stroke, dementia, Huntington's disease, Pick's disease, and Creutzfeid-Jakob disease since it can control simultaneously the production and inhibition of glutamate generated from NAAG hydrolysis and causing neurotoxicity.

The present invention also provides a method for the treatment of disease selected from the group consisting of amyloidosis, Alzheimer's disease, Down syndrome accompanying Alzheimer's disease, stroke, dementia, Huntington's disease, Pick's disease, and Creutzfeid-Jakob disease, which includes the step of administering a pharmaceutically effective dose of the GCP II mutant to a subject having the disease selected from the group consisting of amyloidosis, Alzheimer's disease, Down syndrome accompanying Alzheimer's disease, stroke, dementia, Huntington's disease, Pick's disease, and Creutzfeid-Jakob disease.

The present invention also provides a method for the prevention of disease selected from the group consisting of amyloidosis, Alzheimer's disease, Down syndrome accompanying Alzheimer's disease, stroke, dementia, Huntington's disease, Pick's disease, and Creutzfeid-Jakob disease, which includes the step of administering a pharmaceutically effective dose of the GCP II mutant to a subject.

The present invention further provides a pharmaceutical composition for the screening of a preventive and therapeutic agent for the disease selected from the group consisting of amyloidosis, Alzheimer's disease, Down syndrome accompanying Alzheimer's disease, stroke, dementia, Huntington's disease, Pick's disease, and Creutzfeid-Jakob disease that comprises GCP II mutant gene prepared by replacing the 699^{th} amino acid from N-terminal of the total amino acid sequence of GCP II (Lysine, K) with Serine (S) as an active ingredient.

The GCP II mutant gene preferably has the nucleotide sequence represented by SEQ ID. NO: 1, but not always limited thereto.

The GCP II (glutamate carboxypeptidase II) mutant K699S of the present invention not only has excellent Aβ cleavage activity but also overcomes the stability problem that the wild type GCP II has, so that the composition comprising the GCP II mutant gene as an active ingredient can be effectively used as a pharmaceutical composition for the screening of a preventive and therapeutic agent for the disease selected from the group consisting of amyloidosis, Alzheimer's disease, Down syndrome accompanying Alzheimer's disease, stroke, dementia, Huntington's disease, Pick's disease, and Creutzfeid-Jakob disease since it can control simultaneously the production and inhibition of glutamate generated from NAAG hydrolysis and causing neurotoxicity.

The present invention further provides a pharmaceutical composition for the screening of a preventive and therapeutic agent for the disease selected from the group consisting of amyloidosis, Alzheimer's disease, Down syndrome accompanying Alzheimer's disease, stroke, dementia, Huntington's disease, Pick's disease, and Creutzfeid-Jakob disease that comprises GCP II mutant protein prepared by replacing the 699^{th} amino acid from N-terminal of the total amino acid sequence of GCP II (Lysine, K) with Serine (S) as an active ingredient.

The GCP II mutant protein has preferably the amino acid sequence represented SEQ ID. NO: 2, but not always limited thereto.

The GCP II (glutamate carboxypeptidase II) mutant K699S of the present invention not only has excellent Aβ cleavage activity but also overcomes the stability problem that the wild type GCP II has, so that the composition comprising the GCP II mutant protein as an active ingredient can be effectively used as a pharmaceutical composition for the screening of a preventive and therapeutic agent for the disease selected from the group consisting of amyloidosis, Alzheimer's disease, Down syndrome accompanying Alzheimer's disease, stroke, dementia, Huntington's disease, Pick's disease, and Creutzfeid-Jakob disease since it can control simultaneously the production and inhibition of glutamate generated from NAAG hydrolysis and causing neurotoxicity.

The present invention also provides a screening method of a candidate material for the prevention and treatment of disease selected from the group consisting of amyloidosis, Alzheimer's disease, Down syndrome accompanying Alzheimer's disease, stroke, dementia, Huntington's disease, Pick's disease, and Creutzfeid-Jakob disease comprising steps of:

1) treating test samples to the cells expressing GCP II mutant prepared by replacing the 699^{th} amino acid from N-terminal of the total amino acid sequence of GCPII (Lysine, K) with Serine (S);

2) measuring the expression level of GCP II mutant protein in the cells of step 1); and

3) selecting a test sample that reduces the expression level of GCP II mutant protein of step 2), compared with the control group not treated with any test sample.

In the above method, the expression level of the protein of step 2) can be measured by any one of the methods selected from the group consisting of immunofluorescence method, ELISA, western blotting, and RT-PCR, but not always limited thereto.

The GCP II (glutamate carboxypeptidase II) mutant K699S of the present invention not only has excellent Aβ cleavage activity but also overcomes the stability problem that the wild type GCP II has, so that the expression level of the GCP II mutant protein can be effectively used for the screening of a preventive and therapeutic agent for the disease selected from the group consisting of amyloidosis, Alzheimer's disease, Down syndrome accompanying Alzheimer's disease, stroke, dementia, Huntington's disease, Pick's disease, and Creutzfeid-Jakob disease since it can control simultaneously the production and inhibition of glutamate generated from NAAG hydrolysis and causing neurotoxicity.

In addition, the present invention provides a screening method of a candidate material for the prevention and treatment of disease selected from the group consisting of amyloidosis, Alzheimer's disease, Down syndrome accompanying Alzheimer's disease, stroke, dementia, Huntington's disease, Pick's disease, and Creutzfeid-Jakob disease comprising steps of:

1) treating test samples to GCPII mutant protein prepared by replacing the 699^{th} amino acid from N-terminal of the total amino acid sequence of GCP II (Lysine, K) with Serine (S)

2) measuring the activity of GCPII mutant protein of step 1); and

3) selecting a test sample that reduces the activity of GCP II mutant protein of step 2), compared with the control group not treated with any test sample.

In the above method, the activity of the protein of step 2) can be measured by any one of the methods selected from the group consisting of immunofluorescence method, ELISA, mass spectrometry, and protein chip, but not always limited thereto.

The GCP II (glutamate carboxypeptidase II) mutant K699S of the present invention not only has excellent Aβ cleavage activity but also overcomes the stability problem that the wild type GCP II has, so that the activity of the GCP II mutant protein can be effectively used for the screening of a preventive and therapeutic agent for the disease selected from the group consisting of amyloidosis, Alzheimer's disease, Down syndrome accompanying Alzheimer's disease, stroke, dementia, Huntington's disease, Pick's disease, and Creutzfeid-Jakob disease since it can control simultaneously the production and inhibition of glutamate generated from NAAG hydrolysis and causing neurotoxicity.

In the screening method of the present invention, the interaction between the composition comprising GCP II mutant gene and a test sample can be identified by any conventional method used for the confirmation of such reactions as DNA-DNA, DNA-RNA, DNA-protein, and DNA-compound.

For example, any method to measure the gene expression such as hybridization to confirm the conjugation of the target gene and a test sample in vitro, northern blot assay performed after reacting mammalian cells with a test sample, quantitative PCR, and quantitative real-time PCR; and any method to measure the reporter protein expression after linking a reporter gene to the target gene to be introduced in cells and then inducing reaction with a test sample can be used herein.

At this time, the composition of the present invention can include distilled water or buffer solution to maintain the nucleic acid structure stable, in addition to the GCP II mutant gene.

In the screening method of the present invention, interaction between the composition containing the GCP II mutant gene and a test sample can be identified by any conventional method used for the confirmation of such reactions as protein-protein, and protein-compound.

For example, a method to measure the activity after inducing reaction between the GCP II mutant gene or GCP II mutant protein and a test sample; yeast two-hybrid; screening of phage-displayed peptide clone that is binding to the GCP II mutant protein; HTS (high throughput screening) using natural and chemical library; drug hit HTS; cell-based screening; or DNA microarray can be used for the screening.

At this time, the composition of the present invention can include buffer or reaction solution to maintain the protein structure or its physiological activity stable, in addition to the protein expressed from the GCP II mutant. For in vivo test, the composition of the present invention can additionally include cells expressing the said protein or cells comprising the plasmid expressing the said protein in the presence of a promoter that can regulate the transcription rate.

In the screening method of the present invention, the test sample is presumed to be the possible preventive and therapeutic agent for the control of Aβ accumulation in the brain or the production of glutamate therein, but any randomly selected nucleic acid, protein, other extracts, or natural substance can be the one.

Practical and presently preferred embodiments of the present invention are illustrative as shown in the following Examples, Experimental Examples and Manufacturing Examples.

However, it will be appreciated that those skilled in the art, on consideration of this disclosure, may make modifications and improvements within the spirit and scope of the present invention.

### EXAMPLE 1

### Effect of 2-PMPA on Aβ Cleavage and NAAG Cleavage

### <1-1> Cell Culture and Transfection

HEK293T and H4 cells (American Type Culture Collection, ATCC, Edinburg, Va., USA) were cultured in Dulbecco's modified Eagle's Medium (DMEM; GIBCO) supplemented with 10% fetal bovine serum (FBS; GIBCO) and 1% penicillin/streptomycin (GIBCO) and PC3 cells were cultured in RPMI Medium 1640 (GIBCO) supplemented with 10% fetal bovine serum (FBS; GIBCO) and 1% penicillin/streptomycin (GIBCO) at 37° C. in a humidified incubator containing 5% CO₂. Transient transfections were performed using Lipofectamine 2000 reagent or Lipofectamine Plus reagent (Invitrogen) following manufacturer's protocol.

### <1-2> Preparation of Aβ Peptide

Aβ₁₋₄₀ or Aβ₁₋₄₂ (Invitrogen) peptide was prepared with cold hexafluoroisopropanol (HFIP; Sigma) at the final concentration of 1 mM. After the shaking for 40 min at RT and standing for 20 min at 4° C., HFIP was removed under vacuum. The peptide pellets were stored at −70° C. The pellets were dissolved in DMSO to make the final concentration 5 mM and then distilled water was added until the concentration was adjusted to 1 mM.

### <1-3> Investigation of the Effect of 2-PMPA on Aβ Cleavage by ELISA

Vectors encoding the hGCP II and various mutant hGCP II genes were transfected into PC3 cells in a 12-well plate. About 30 hr later, the culture medium was changed with fresh one treated with Aβ₁₋₄₀ of Aβ₁₋₄₂ peptide in the presence or absence of 2-PMPA, and the cells were incubated for another 8 hr. The medium was collected and assayed for residual Aβ₁₋₄₀ or Aβ₁₋₄₂ using ELISA kits (Invitrogen) following manufacturer's protocol. Cells transfected with the empty pcDNA3 empty vector were used as the negative control.

As a result, as shown in FIG. 1a, it was confirmed that rhGCP II cleaved Aβ₁₋₄₀ peptide about 80 percent compared with control, but 2-PMPA had no effect on Aβ₁₋₄₀ cleavage of rhGCP II (FIG. 1a).

### <1-4> Confirmation of the Effect of 2-PMPA on NAAG Cleavage by NAAG Cleavage Assay

The activity of endogenous or over-expressed Glutamate carboxypeptidase II protein (GCP II) was measured as follows. Cell lysate (50 μg) was incubated with 20 μM of N-acetyl-L-aspartyl-L-[3,4-3H] glutamate (NAAG; NEN corporation) with or without 20 nM 2-PMPA, a GCP II specific inhibitor, in a total volume of 100 μl containing 50 mM HEPES and 150 mM NaCl for 1 hr at 37° C. Upon completion of the reaction, sample mixture was loaded on AG 1-X8 anion-exchange resin (Biorad) prepared in 96 well columns, followed by centrifugation at 2000 rpm for 5 min. The resin bound with the sample mixture was eluted with 0.5 mM formate (100 ml) and centrifuged at 2000 rpm for 5 min. All the eluted samples were mixed with 1 ml of scintillation solution (Optiphase HiSafe; Wallac) and then radioactivity was measured by scintillation counter (Wallac Inc.).

As a result, as shown in FIG. lb, it was

confirmed that hydrolysis of NAAG by GCP II was completely blocked by 2-PMPA (FIG. 1b).

### <1-5> Confirmation of the Effect of 2-PMPA on Aβ Cleavage Activity of Lentiviral GCP II by Using the Prepared GCP II Plasmid and Lentivirus

The hGCP II plasmids were prepared as follows. RNA was extracted from U87-MG cells, the human astrocytes, and then cDNA was synthesized by using reverse transcriptase. For the amplification of hGCP II from the synthesized cDNA, PCR was performed by using the following primers. The PCR product was cloned into pcDNA3 vector to prepare the plasmid:

Forward:
(SEQ ID. NO: 3)
5′-GATGTGGAATCTCCTTCACGAAAC-3′;
and
Reverse:
(SEQ ID. NO: 4)
5′-ATCCTCTTAGGCTACTTCACTCAAAG-3′.

Lentiviral hGCP II was prepared by Macrogen Inc. (Seoul, Korea). Briefly, hGCP II cDNA was cloned into a lentiviral construct (LentiM1.4-hGCP II) containing the human cytomegalovirus (CMV) promoter and IRES sequence with fluorescent protein (GFP). For virus production, the hGCP II, VSV-G, and gag-pol expression vectors were cotransfected into 293T cells using Lipofectamine Plus (Invitrogen, Carlsbad, Calif., USA). The cells were treated with Aβ monomers and 2-PMPA. The concentration of Aβ₁₋₄₀ in the cell growth medium was measured by the same manner as used in Example <1-3>.

As a result, as shown in FIG. 1c, it was confirmed that 2-PMPA did not block A₁₋₄₀ cleavage of lentiviral GCP II (FIG. 1c).

### <1-6> Transgenic Animal

Double-transgenic APP Swedish/PS1ΔE9 mice were purchased from the Jackson Laboratory (Bar Harbor, Me., USA) and maintained as double hemizygotes by crossing with wild-type mice on C57BL/6J background strain. All the animals were housed according to standard animal care protocols and maintained in a pathogen-free facility at the Korean Food and Drug Administration. Mouse genotypes were confirmed by PCR with the following primers:

PrP (mouse prion protein):
Forward:
(SEQ ID. NO: 5)
5′-CCTCTTTGTGACTATGTGGACTGATGTCGG-3′
Reverse:
(SEQ ID. NO: 6)
5′-GTGGATAACCCCTCCCCCAGCCTAGACC-3′;
Human APP:
Forward:
(SEQ ID. NO: 7)
5′-GACTGACCACTCGACCAGGTTCTG-3′;
and
Reverse:
(SEQ ID. NO: 8)
5′-CTTGTAAGTTGGATTCTCATATCCG-3′.

To examine the effect of a GCP II inhibitor on the brain Aβs level, the experimental group was treated with 2-PMPA (10 mg/kg) dissolved in phosphate-buffered saline (PBS) 2×/wk for 1 month by intraperitoneal injection. The untreated group was administered with an equal volume of PBS as control. Treatment was started when the mice were 8 month old.

### <1-7> Confirmation of the Effect of 2-PMPA in the Mouse Brain

To investigate the effect of 2-PMPA in the mouse brain, 8-month old APP Swedish/PS1Δ9 mice transfected by the same manner as described in Example <1-6> were treated nine times with 2-PMPA (10 mg/kg, intraperitoneally, 2/wk for 1 month) or the same amount of PBS. Aβp₁₋₄₀ and Aβ₁₋₄₂ levels in the cortical membrane fractions were analyzed by the same manner as described in Example <1-3>. NAAG cleavage activity of GCP II was measured by the same manner as described in Example <1-4>.

As a result, as shown in FIG. 1d, NAAG cleavage activity shown in the mouse treated with 2-PMPA was approximately 90% decreased, compared with that of the control treated with PBS alone (FIG. 1d). However, as shown in FIG. 1e, total levels of Aβ₁₋₄₀ and Aβ₁₋₄₂ were not changed by the treatment of 2-PMPA (FIG. 1e).

### EXAMPLE 2

### Site-Directed Mutagenesis of GCP II and Expression of GCP II Mutants

### <2-1> Site-Directed Mutagenesis of GCP II

The pcDNA-hGCP II plasmid (50 ng) was used as a template, and each mutation was introduced by two complementary oligonucleotide primers harboring the desired mutation. Using 2.5 U of pfu Ultra High-Fidelity DNA polymerase (Stratagene), extension and incorporation of the mutagenic primers (125 ng) were performed, resulting in nicked circular strands (95° C., 5 min→(95° C., 50 sec→55° C., 1 min→80° C., 8 min 20 sec→72° C., 1 min)×22 cycles→4° C., ∞). The methylated, nonmutated parental DNA template was digested by 20 U of Dpn I (NEB) for 1 hr at 37° C. Then, the circular dsDNA was transformed into DH5a competent cells. The presence of individual mutations was confirmed by sequencing (Cosmo corporation).

As a result, as shown in FIG. 2a, 7 GCP II mutants (R210A, D387N, P388A, R536L, G548P, Y552I, and K699S) were prepared (FIG. 2a).

### <2-2> Confirmation of GCP II Mutant Expression by Western Blotting

To confirm the GCP II mutant expression, mutant plasmids were transfected into HEK293T cells by the same manner as described in Example <1-1>. Cells were washed with phosphate buffered saline (PBS; GIBCO) and lysed in RIPA buffer (150 mM NaCl, 1% NP-40, 0.5% sodium deoxycholate, 0.1% SDS, 50 mM Tris at pH 8.0) with protease inhibitor cocktail (Sigma). The lysate was analyzed by Bradford assay to measure the protein concentration. The same concentration of proteins were added with β-mercaptoethanol and boiled for 10 min and separated by 10% SDS-PAGE. Proteins were electrotransferred onto polyvinylidene difluoride (PVDF) membrane (GE Healthcare) and blocked with 5% nonfat dry milk in TBS-T at room temperature for 1 hr. Then, the membrane was incubated with anti-PSMA (abeam; ab41034, 1:1000), Y-PSMA (Maine Biotech, 1:1000) or anti-α-tubulin antibody (Sigma-Aldrich; T6199, 1:50000) in 5% nonfat dry milk at 4° C. for overnight. After washing with TBS-T three times, the blots were incubated with HRP-conjugated goat anti-mouse IgG (Jackson IR; 115-035-071, 1:10000) or HRP-conjugated goat anti-rabbit IgG (Jackson IR; 115-035-046, 1:10000) for 2 hrs at room temperature. After washing three times, proteins were detected using chemiluminescent substrate (Thermo Scientific).

As a result, as shown in FIG. 2b, the pattern of bands was similar with that of rhGCP II and one of the bands was predominately seen at 98 kDa which is the predicted size of GCP II (FIG. 2b).

### EXAMPLE 3

### Effect of GCP II mutation on NAAG cleavage

To investigate the effect of GCP II mutation on NAAG cleavage, mutant proteins with a substitution in the S1′ pocket (R210A, K699S) and SI pocket (R536L, G548P) were transfected in H4 cells by the same manner as described in Example <1-1>. To measure NAAG hydrolysis, cell lysate was incubated with [H]³-labeled NAAG (20 μM) and

specific substrate of GCP II by the same manner as described in Example <1-4>. Protein expression was examined by the same manner as described in Example <2-2>.

As a result, as shown in FIG. 3a, the S1′ pocket mutants (R210A, K699S) completely lost the activity of NAAG hydrolysis, while the SI pocket mutants (R536L, G548P) maintained the activity of NAAG hydrolysis approximately 40% by that of the wild type GCP II (FIG. 3a).

To investigate the effect of Aβ on NAAG cleavage of GCP II, rhGCP II was incubated with [H]³-labeled NAAG (20 μM) and A(3 peptides by the same manner as described in Example <1-4>.

As a result, as shown in FIG. 3b, it was confirmed that NAAG cleavage activity of GCP II was not affected by Aβ peptide (FIG. 3b).

### EXAMPLE 4

### Effect of GCP II Mutation on Aβ Cleavage

### <4-1> GCP II/Aβ Complex Modeling

The mode of binding of Aβ-S1 and S1′ pockets were modeled using program O (http://xray.bmc.uu.se/alw yn/A-Z frameset.html) (PDB file: 1z0q (Aβ), 2oot (GCP II)). Computational docking was performed using manual docking method.

To identify Aβ clearance activity of GCP II mutants, various mutant plasmids were transfected in PC3 cells known as not to express endogenous GCP II by the same manner as described in Example <1-5> and in Example <4-1>. 48 hrs later, the cell lysate was mixed with Aβ (2 μM) and incubated for overnight.

### <4-2> Confirmation of the Effect of GCP II Mutation on Aβ Cleavage by Dot Blotting

To quantify the remaining Aβ peptides, the mixture of cell lysate and Aβ (2 μM) proceeded to dot blotting and the result was analyzed by the same manner as described in Example <2-2>. 1 μl of the mixture of cell lysate and Aβ (2 μM) was dropped on nitrocellulose membrane, which was then completely dried. The dried membrane proceeded to blocking with TBS-T containing 5% nonfat dry milk at room temperature for 1 hr. Then, the membrane was incubated with anti-Aβ antibody, 6E10 (SIGNET; SIG-39300, 1:2000) in 5% nonfat dry milk at 4° C. for overnight. After washing with TBS-T three times, the membrane was incubated with HRP-conjugated goat anti-mouse IgG (Jackson IR; 115-035-071, 1:10000) for 1 hr at room temperature. After washing three times, proteins were detected using chemiluminescent substrate.

As a result, as shown in FIGS. 4a and 4b, the K699S mutant showed higher Aβ cleavage activity than the wild type, while the G548P mutant lost Aβ cleavage activity (FIGS. 4a and 4b).

To confirm cleavage activity of GCP II on exogenously treated Aβ peptides, one day after transfection, 1 ng/ml of Aβ was treated into the medium, followed by incubation at 37° C. for 8 hrs by the same manner as described in Examples <1-3> and <4-1>.

As a result, as shown in FIG. 4c, the residual Aβ was reduced by K699S but not by G548P (FIG. 4c).

### INDUSTRIAL APPLICABILITY

As explained hereinbefore, the present invention can be effectively used for the development of a medicine for the prevention and treatment of disease selected from the group consisting of amyloidosis, Alzheimer's disease, Down syndrome accompanying Alzheimer's disease, stroke, dementia, Huntington's disease, Pick's disease, and Creutzfeid-Jakob disease, and the development of a method for the prevention and treatment of the said disease by using the same, which is precisely exemplified by protein therapy, expression/activity regulator, gene therapy, and cell therapy.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

What is claimed is:

1. A method for treating disease comprising administering a pharmaceutical composition that comprise GCP II mutant prepared by replacing the 699^{th} amino acid from N-terminal of the total amino acid sequence of GCP II (glutamate carboxypeptidase II) (Lysine, K) with Serine (S) as an active ingredient to a subject with disease selected from the group consisting of amyloidosis, Alzheimer's disease, Down syndrome accompanying Alzheimer's disease, stroke, dementia, Huntington's disease, Pick's disease, and Creutzfeid-Jakob disease.

2. The method of claim 1, wherein the GCP II mutant characteristically has the amino acid sequence represented by SEQ ID. NO: 2.

3. The method of claim 1, wherein the GCP II mutant characteristically has the inhibitory activity of glutamate production and the β-amyloid cleavage activity at the same time.

4. The method of claim 3, wherein the β-amyloid is characteristically soluble or insoluble β-amyloid.

5. A method for treating disease comprising administering a pharmaceutical composition that comprise the expression vector expressing the GCP II mutant prepared by replacing the 699^{th} amino acid from N-terminal of the total amino acid sequence of GCP II (Lysine, K) with Serine (S) as an active ingredient to a subject with disease selected from the group consisting of amyloidosis, Alzheimer's disease, Down syndrome accompanying Alzheimer's disease, stroke, dementia, Huntington's disease, Pick's disease, and Creutzfeid-Jakob disease.

6. A method of claim 5, wherein the expression vector is a viral expression vector selected from the group consisting of adenovirus, adeno-associated virus, retrovirus, and vaccinia virus.

7. A screening method of a candidate material for the prevention and treatment of disease selected from the group consisting of amyloidosis, Alzheimer's disease, Down syndrome accompanying Alzheimer's disease, stroke, dementia, Huntington's disease, Pick's disease, and Creutzfeid-Jakob disease comprising steps of:
i) (a) treating test samples to the cells expressing GCP II mutant prepared by replacing the 699^{th} amino acid from N-terminal of the total amino acid sequence of GCP II (Lysine, K) with Serine (S), or (b) treating test samples to GCP II mutant protein prepared by replacing the 699^{th} amino acid from N-terminal of the total amino acid sequence of GCP II (Lysine, K) with Serine (S);
ii) (a) measuring the expression level of GCP II mutant protein in the cells of step 1) or (b) measuring the activity of GCP II mutant protein of step 1); and
iii) (a) selecting a test sample that reduces the expression level of mutant protein of step 2), compared with the control group not treated with any test sample or (b) selecting a test sample that reduces the activity of GCP II mutant protein of step 2), compared with the control group not treated with any test sample.

8. The method of claim 7, wherein the expression level of the protein of step ii) (a) is measured by any one of the methods selected from the group consisting of immunofluorescence method, ELISA, western blotting, and RT-PCR.

9. The screening method of claim 7, wherein the activity of the protein of step ii) (b) is measured by any one of the methods selected from the group consisting of immunofluorescence method, ELISA, mass spectrometry, and protein chip.
